SEQUENCE LISTING (5 sequences)

SEQ ID NO: 1 - protein, 240 aa, Homo sapien
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 2 - protein, 5 aa, Artificial Sequence
  fragments of CD7
VLVRT

SEQ ID NO: 3 - DNA, 15 bp, Artificial Sequence
  fragments of CD7
gtgctggtgaggaca

SEQ ID NO: 4 - DNA, 15 bp, Artificial Sequence
  fragments of CD7
gtgctggcgaggaca

SEQ ID NO: 5 - protein, 5 aa, Artificial Sequence
  fragments of CD7
VLART

(19) United States
(12) Patent Application Publication
(10) Pub. No.: US 2005/0277587 A1
(43) Pub. Date: Dec. 15, 2005

(54) CD7 as biomarker and therapeutic target for psoriasis

(75) Inventors: Yuan-Tsong Chen, Taipei City (TW); Jer-Yuarn Wu, Taichung (TW); Whu-Liang Hwu, Taipei (TW); Cathy S. J. Fann, Taipei (TW); Chi-Fan Yang, Taipei (TW)

Correspondence Address: FISH & RICHARDSON P.C., PO BOX 1022, MINNEAPOLIS, MN, 55440-1022, US

(73) Assignee: Academia Sinica, Nan-Kang (TW)

(21) Appl. No.: 10/866,530

(22) Filed: Jun. 10, 2004

(51) Int. Cl. A61K 38/17; C07K 14/74; C12Q 1/68; C07H 21/04

(52) U.S. Cl. 514/12; 435/6; 435/69.1; 435/320.1; 435/325; 530/350; 536/23.5

(57) ABSTRACT

We present information obtained from the occurrence of psoriasis and its related disease pityriasis rubra pilaris (PRP) in a large, five-generation kindred. Using a genome-wide scan and single nucleotide polymorphism (SNP) fine mapping, the psoriasis/PRP locus was mapped to chromosome 17q terminus, a region close to but distinct from the 17q PSOR2 locus. Moreover, we sequenced the candidate genes in this region, and identified CD7 as the susceptibility gene in this family. Compositions and methods of use are provided for the prediction, diagnosis, disease monitoring and therapeutic development of psoriasis and pityriasis rubra pilaris (PRP).

### TECHNICAL FIELD

This invention pertains to loci and genes associated with psoriasis and pityriasis rubra pilaris, as well as related compositions and uses thereof.

### REFERENCES

The following publications, patent applications, and patents are cited in this application:

Bartolini, F., et al. (2002). Functional overlap between retinitis pigmentosa 2 protein and the tubulin-specific chaperone cofactor C. J Biol Chem. 277(17):14629-14634.

Bhamidipati, A, et al. (2000). ADP ribosylation factor-like protein 2 (Ar12) regulates the interaction of tubulin-folding cofactor D with native tubulin. J Cell Biol. 149(5):1087-1096.

Bjerke, J. R., et al. (1978). Characterization of mononuclear cell infiltrates in psoriatic lesions. J Invest Dermatol. 71(5):340-343.

Capon, F., et al. (2001). Fine mapping of the PSORS4 psoriasis susceptibility region on chromosome 1q21. J Invest Dermatol. 116(5):728-730.

Carrera, A. C., Rincon, M., Sanchez-Madrid, F., Lopez-Botet, M. and de Landazuri, M. O. (1988) Triggering of co-mitogenic signals in T cell proliferation by anti-LFA-1 (CD18, CD11a), LFA-3, and CD7 monoclonal antibodies. J. Immunol. 141: 1919-1924.

Chan, A. S. H., Mobley, J. L., Fields, G. B. and Shimizu, Y. (1997) CD7-mediated regulation of integrin adhesiveness on human T cells involves tyrosine phosphorylation-dependent activation of phosphatidyinositol 3-kinase. J. Immunol. 159: 934-942.

Elder, J. T., et al. (2001). The genetics of psoriasis 2001: the odyssey continues. Arch Dermatol. 137(11):1447-1454.

Enlund, F., et al. (1999a). Analysis of three suggested psoriasis susceptibility loci in a large Swedish set of families: confirmation of linkage to chromosome 6p (HLA region), and to 17q, but not to 4q. Hum Hered. 49(1):2-8.

Enlund, F., et al. (1999b). Psoriasis susceptibility locus in chromosome region 3q21 identified in patients from southwest Sweden. Eur J Hum Genet. 7(7):783-790.

Fukumori, T., Takenaka, Y, Yoshii, T., Kim, H. R., Hogan, V., Inohara, H., Kagawa, S., Raz, A. (2003) CD29 and CD7 mediate galectin-3-induced type II T-cell apoptosis. Cancer Res. 63:8302-11.

Gottlieb, A. B., and Krueger, J. G. (1990). HLA region genes and immune activation in the pathogenesis of psoriasis. Arch Dermatol. 126(8):1083-1086.

Gottlieb, S. L., et al. (1995). Response of psoriasis to a lymphocyte-selective toxin (DAB389IL-2) suggests a primary immune, but not keratinocyte, pathogenic basis. Nat Med. 1(5):442-447.

Harlow and Lane (1989). Antibodies: a laboratory manual. Cold Spring Harbor Laboratory, New York, N.Y.

Helms, C., et al. (2003). A putative RUNX1 binding site variant between SLC9A3R1 and NAT9 is associated with susceptibility to psoriasis. Nat Genet. 35(4):349-356.

Iizuka, M., et al. (1992). Cloning and polymerase chain reaction-single-strand conformation polymorphism analysis of anonymous Alu repeats on chromosome 11. Genomics 12(1):139-146.

Iwahana, H., et al. (1995). Multiple fluorescence-based PCR-SSCP analysis with postlabeling. PCR Methods Appl. 4(5):275-282.

Jung, L. K., Roy, A. K., Chakkalath, H. R. (1992) CD7 augments T cell proliferation via the interleukin-2 autocrine pathway. Cell. Immunol. 141: 189-199.

Lazarovits, A. I., Osman, N., LeFeuvre, C. E., Ley, S C. and Crumpton, M. J. (1994) CD7 is associated with CD3 and CD45 on human T cells. J. Immunol. 153: 3956-3966.

Lebwohl, M. (2003). Psoriasis. Lancet 361(9364):1197-1204.

Ledbetter, J. A., June, C. H., Grosmaire, L. S. and Rabinovitch, P. S. (1987) Cross-linking of surface antigens causes mobilization of intracellular ionized calcium in T lymphocytes. Proc. Natl. Acad. Sci. USA 84: 1384-1388.

Lee, Y. A., et al. (2000). Genomewide scan in german families reveals evidence for a novel psoriasis-susceptibility locus on chromosome 19p13. Am J Hum Genet. 67(4):1020-1024.

Lyman, S. D., Escobar, S., Rousseau, A. M., Armstrong, A., Fanslow, W. C. (2000) Identification of CD7 as a cognate of the human K12 (SECTM1) protein. J Biol Chem. 275:3431-7.

Martin, E. R., et al. (2001). Correcting for a potential bias in the pedigree disequilibrium test. Am J Hum Genet. 68(4):1065-1067.

Mashiyama, S., et al. (1991). Detection of p53 gene mutations in human brain tumors by single-strand conformation polymorphism analysis of polymerase chain reaction products. Oncogene. 6(8):1313-1318.

Matthews, D., et al. (1996). Evidence that a locus for familial psoriasis maps to chromosome 4q. Nat Genet. 14(2):231-233.

Nair, R. P., et al. (1997). Evidence for two psoriasis susceptibility loci (HLA and 17q) and two novel candidate regions (16q and 20p) by genome-wide scan. Hum Mol Genet. 6(8): 1349-1356.

Nielsen, P. E., et al. (1991). Sequence-selective recognition of DNA by strand displacement with a thymine-substituted polyamide. Science 254(5037):1497-1500.

Roberts, A. A., Amano, M., Felten, C., Galvan, M., Sulur, G., Pinter-Brown, L., Dobbeling, U., Burg, G., Said, J., Baum, L. G. (2003) Galectin-1-mediated apoptosis in mycosis fungoides: the roles of CD7 and cell surface glycosylation. Mod Pathol. 16:543-51.

Speckman, R. A., et al. (2003). Novel immunoglobulin superfamily gene cluster, mapping to a region of human chromosome 17q25, linked to psoriasis susceptibility. Hum Genet. 112(1):34-41.

Stem, R. S. (1997). Psoriasis. Lancet 350(9074):349-353.

Stillwell, R. and Bierer, B. E. (2001) T cell signal transduction and the role of CD7 in costimulation. Immunol. Res. 24: 31-52.

Subrahmanyam, G., et al. (2003). Association of T cell antigen CD7 with type II phosphatidylinositol-4 kinase, a key component in pathways of inositol phosphate turnover. Eur J Immunol. 33(1):46-52.

Subrahmanyam, G., Rudd, C. E., Schneider, H. (2003) Association of T cell antigen CD7 with type II phosphatidylinositol-4 kinase, a key component in pathways of inositol phosphate turnover. Eur J Immunol. 33:46-52.

The International Psoriasis Genetics Study: assessing linkage to 14 candidate susceptibility loci in a cohort of 942 affected sib pairs. (2003) Am J Hum Genet. 73(2):430-437.

Tomfohrde, J., et al. (1994). Gene for familial psoriasis susceptibility mapped to the distal end of human chromosome 17q. Science 264(5162):1141-1145.

Veal, C. D., et al. (2001). Identification of a novel psoriasis susceptibility locus at 1p and evidence of epistasis between PSORS1 and candidate loci. J Med Genet. 38(1):7-13.

All of the publications, patent applications, and patents, cited above or elsewhere in this application, are herein incorporated by reference in their entirety to the same extent as if each individual publication, patent application or patent was specifically and individually indicated to be incorporated by reference in its entirety.

### STATE OF THE ART

Psoriasis is a chronic, inflammatory, hyperproliferative disease of the skin, scalp, nails and joints, which affects up to 2% of adults globally (Stern, 1974; Lebwohl, 2003). The disease varies in severity. Some patients display a mild disease with isolated scaling erythematous plaques on the elbows or knees, whereas others can have most of their cutaneous surface affected. The most common form of psoriasis is plaque psoriasis. At the cellular level, psoriasis is characterized by markedly increased epidermal proliferation and incomplete differentiation, the elongation, dilation, and leakiness of the superficial plexus of dermal capillaries, and a mixed inflammatory and immune cell infiltrate of the epidermis and papillary dermis (Stern, 1974; Lebwohl, 2003). Dermal infiltrates comprised of T cells and macrophages typically appear in early lesions before epidermal changes (Bjerke et al., 1978). The therapeutic effect of the immunosuppressive agents suggests a primary immune pathogenic basis of psoriasis (Gottlieb et al., 1995).

The susceptibility to the development of psoriasis likely has a significant genetic component. Accumulating evidence suggests that psoriasis is a multifactorial disorder caused by the concerted action of multiple disease genes in a single individual, and that the genetic activity is triggered by environmental factors (Elder et al., 2001). Some of these genes control the severity of a variety of diseases, via their regulation of the inflammation and immune processes (severity genes), whereas other genes are unique to psoriasis (specific genes).

A number of genetic studies have sought to identify the psoriasis susceptibility loci. Associations between psoriasis and human lymphocyte antigen (HLA) alleles were first described in 1990 (Gottlieb and Krueger, 1990). Subsequently, genome-wide linkage scans have mapped psoriasis to several chromosomal regions including PSORS1 at 6p21,7 (Enlund et al., 1999), PSORS2 at 17q (Nair et al., 1997; Enlund et al., 1999a; Tomfohrde et al., 1994), PSORS3 at 4q (Matthews et al., 1996), PSORS4 at 1q, (Capon et al., 2001), PSORS5 at 3q (Enlund et al., 1999b), PSORS6 at 19p (Lee et al., 2000), and PSORS7 at 1p (Veal et al., 2001). Recently, the International Psoriasis Genetics Consortium reassessed these candidate loci using a cohort of 942 affected sib pairs. This reassessment confirmed the significant linkage on chromosomes 6p21, 16q and 10q22-q23 (The International Psoriasis Genetics Study, 2003). However, PSORS2 on chromosome 17q24-25 did not achieve a maximum LOD score greater than 0.9 in that study, even though this region has been implicated in psoriasis in several families studies (Nair et al., 1997; Enlund et al., 1999a; Tomfohrde et al., 1994). Interestingly, a RUNX1 binding site variant located in the 17q25 region, between SLC9A3R1 and NAT9, has recently been identified as the first putative susceptibility gene for psoriasis (Helms et al., 2003). Therefore, the importance of the 17q25 region remains controversial. In addition, although a number of loci have been associated with psoriasis, more susceptibility genes for psoriasis need to be identified in order to understand the molecular mechanism and pathogenesis of the disease.

Pityriasis rubra pilaris (PRP) is a chronic papulosquamous disorder of unknown etiology characterized by reddish orange scaly plaques, palmoplantar keratoderma, and keratotic follicular papules. The disease may progress to erythroderma with distinct areas of uninvolved skin, the so-called islands of sparing. The gene for PRP has not been found.

### SUMMARY

We present information obtained from the occurrence of psoriasis/pityriasis rubra pilaris (PRP) in a large, five-generation kindred. In this family, the severity of the disease varied, ranging from mild skin lesions resembling PRP to severe classical psoriasis. Using a genome-wide scan and single nucleotide polymorphism (SNP) fine mapping, the psoriasis/PRP locus was mapped to chromosome 17q terminus, a region close to but distinct from the 17q PSOR2 locus. Moreover, we sequenced the candidate genes in this region, and identified CD7 as the susceptibility gene in this family. A C to T mutation was found in the CD7 cDNA in all the affected family members, but not in the unaffected family members or control subjects. This point mutation changes amino acid 201 from alanine to valine, which is located in the predicted transmembrane domain of the CD7 molecule.

Accordingly, one aspect of the present invention provides CD7 as a gene for psoriasis or its related disease pityriasis rubra pilaris (PRP). Also provided is a mutant CD7, or a fragment thereof, that is associated with psoriasis/PRP. The mutant CD7 preferably has a mutation at residue 201. The mutation at residue 201 is preferably an amino acid substitution, particularly a substitution with valine. A preferred embodiment is an isolated polypeptide comprising SEQ ID NO:1, except that amino acid residue 201 of SEQ ID NO:1 is mutated. Other preferred polypeptides comprise amino acid residues 26-240, 133-240, 160-240 or 195-240 of SEQ ID NO:1, particularly with residue 201 being mutated. Preferred embodiments also include polypeptides that consist of amino acid residues 1-240, 26-240 and 133-240, respectively, preferably with the mutation at residue 201.

The mutant CD7 may be chemically modified, such as glycosylated or phosphorylated. Fusion proteins of the mutant CD7 are also provided.

Another aspect of the present invention provides antibodies that recognize the mutant CD7 but not the wild-type. The antibody may be monoclonal or polyclonal. The antibody may be derived from any animal that possesses an immune system, including human. The antibody may also be recombinantly prepared or humanized.

Another aspect provides isolated nucleic acids that encode the mutant polypeptides of the present invention. The nucleic acid may be a linear fragment, plasmid, cosmid, yeast artificial chromosome, or any other form of isolated nucleic acid. Preferably, the nucleic acid is an expression vector. Cells comprising the nucleic acid are also provided.

Yet another aspect of the present invention provides an oligonucleotide that hybridizes to a mutated human CD7 gene but not the wild-type human CD7 gene. The mutation is preferably at residue 201, more preferably an amino acid substitution, particularly a substitution with valine. A preferred oligonucleotide comprises a sequence that encodes Leu-Val-Arg (such as CTGGTGAGQ SEQ ID NO:2), or the complement thereof. The oligonucleotide more preferably comprises a sequence that encodes Val-Leu-Val-Arg-Thr (such as GTGCTGGTGAGGACA, SEQ ID NO:3), or the complement thereof.

The oligonucleotide may be 20 nucleotides or less in length. The oligonucleotide may also be 30, 40, 50, or 60 nucleotides in length or less. Preferred ranges of length for the oligonucleotide include 8-20, 10-30, 15-40 and 50-80, in particular 12-20.

Other aspects of the present invention provide kits that comprise the oligonucleotides, mutant CD7 polypeptides, or nucleic acids encoding the mutant polypeptides. Arrays of oligonucleotides comprising the oligonucleotides described above are also provided.

Another aspect of the present invention provides a method for assessing the risk for developing psoriasis or pityriasis rubra pilaris (PRP) in a subject, comprising examining the CD7 gene or gene product of the subject, wherein a mutation of CD7 is indicative of a risk for developing psoriasis or PRP. The mutation is preferably at amino acid residue 201. The method can also be employed for diagnosis of psoriasis or PRP, or disease monitoring thereof. The CD7 gene or gene product can be examined by using any method. For example, genomic DNA can be obtained from the subject, e.g., from blood cells, and the sequence of CD7 gene investigated. A number of techniques are available, including direct sequencing, PCR, single-strand conformation polymorphism, denaturant gradient gel electrophoresis, and allele specific oligonucleotide hybridization, for mutational analyses.

A further aspect of the present invention provides a method for developing a therapy for psoriasis or pityriasis rubra pilaris (PRP), comprising screening candidate medicines using an assay in which CD7 or a CD7 mutant is the target. For example, a cell overexpressing CD7 or the CD7 mutant can be used in an assay to screen for drugs that can stimulate or block CD7 activity. As another example, a cell comprising the CD7 mutant can be used in an assay to screen for drugs that can bind the CD7 mutant, inhibit the CD7 mutant function, or restore the function of the CD7 mutant to a normal level.

Another aspect of the present invention provides a method of pharmacogenomics profiling comprising determining the presence of a CD7 mutation that is associated with psoriasis or pityriasis rubra pilaris (PRP). The method can optionally comprise the determination of other genetic factors. Those other genetic factors may be associated with the predisposition for any disease or medical condition, including psoriasis and PRP. For example, these other genetic factors may be selected from the group consisting of thiopurine methyltransferase and the genes for the long-QT syndrome.

It is contemplated that pathways and/or proteins associated with CD7, particularly the mutant CD7, can also be used in the methods described herein.

The foregoing and other objects, features and advantages of the invention will be apparent from the following more particular description of preferred embodiments of the invention, as illustrated in the accompanying drawings.

### DESCRIPTION OF THE DRAWINGS

FIG. 1 shows the pedigree for a five-generation family with psoriasis/PRP. The arrow indicates the index person (IV-5). Because of the age-dependent variation in the expression of psoriasis, family members aged less than 30 who appeared normal are designated with the “unknown disease” status.

FIG. 2 shows a genome-wide linkage analysis of the extended psoriasis kindred. This analysis includes 43 family members. The Y-axis represents the LOD score calculated using the MLINK program. The X-axis represents the distance from the chromosomal p terminus expressed in Haldane cM.

FIG. 3 shows the two-point LOD scores performed with MLINK crossing chromosome 17. The Y-axis represents the LOD score calculated using the MLINK program. The X-axis represents the distance from the chromosome p terminus expressed in Mb.

FIG. 4 shows the results of a multipoint analysis using LINKMAP program with six additional microsatellite markers in the 17q25 region. The Y-axis represents the LOD score. The X-axis represents the distance in Mb. The LOD scores (in parenthesis) and positions for markers D17S785, D17S784, and D17S928 are also depicted.

FIG. 5 shows the distribution of 202 SNPs used in linkage analyses. The SNPs are depicted according to their relative position to the microsatellite markers D17S785, D17S784 and D17S928.

FIG. 6 shows the results of a multipoint linkage analysis with 45 SNP markers around 17q terminus using the SIMWALK2 program. The Y-axis represents the LOD score. The X-axis represents the distance in kb. SNP#211 has the highest LOD score of 3.404.

FIG. 7 shows the DNA sequence around position 201 of the CD7 gene.

### DETAILED DESCRIPTION

We present information obtained from the occurrence of psoriasis in a large, five-generation kindred. In this family, the severity of the disease varied, ranging from mild skin lesions resembling PRP to severe classical psoriasis. Using a genome-wide scan and single nucleotide polymorphism (SNP) fine mapping, the psoriasis/PRP locus was mapped to chromosome 17q terminus, a region close to but distinct from the 17q PSOR2 locus. Moreover, we sequenced the candidate genes in this region, and identified CD7 as the susceptibility gene in this family. Compositions and methods of use are provided for the prediction, diagnosis, disease monitoring and therapeutic development of psoriasis and pityriasis rubra pilaris (PRP).

Prior to describing the invention in further detail, the terms used in this application are defined as follows unless otherwise indicated.

### DEFINITION

A “drug”, or “medication”, is any compound or material that is administered to a patient for prophylactic, diagnostic or therapeutic purposes.

A subject has a “risk” for a disease if the probability of the subject to develop the disease is higher than the probability of the general population to develop the disease. The probability of the subject to develop the disease is preferably at least about 1.5 fold, more preferably at least about 2 fold, still more preferably at least about 3, 4, 5, 6, 7, 8 or 9 fold, and most preferably at least about 10 fold as high as the probability of the general population to develop the disease.

The term “expressing a polypeptide in a cell” means causing the cell to produce, via protein translation, a polypeptide sequence of interest from a polynucleotide encoding such polypeptide.

An “expression vector” is a vector that encodes one or more polypeptides of interest along with appropriate transcriptional and translational regulatory sequences to allow expression of the polypeptide in a cell. Examples of expression vectors include, but are not limited to, plasmids, phages, yeast artificial chromosomes, and viruses. Expression vectors may comprise inducible or cell-type-specific promoters, enhancers or repressors, introns, polyadenylation signals, selectable markers, polylinkers, site-specific recombination sequences, and other features to improve functionality, convenience of use, and control over mRNA and/or protein expression levels, as known in the art.

An “inducible promoter” is a promoter that causes RNA to be transcribed from a particular polynucleotide sequence at different levels depending upon specific intracellular or environmental conditions. Inducible promoters may respond positively or negatively to, for example, the presence of hormones, nutrients, metabolites, toxins, stress, osmolarity, the activation or inactivation of certain cellular biochemical pathways, or other means of regulating gene expression.

A “polypeptide” is a molecule having any number of contiguous amino acid residues linked via peptide bonds. As used herein, polypeptide and protein are interchangeable terms.

The term “substantial sequence identify”, at the amino acid level, refers to a sequence identity of at least 50%, more preferably at least 60%, 70%, 80%, 85%, 90%, 95%, or 98%. Sequence identity is determined by aligning two sequences of interest by any method established in the art (e.g., BLAST) and determining the percentage of the amino acid residues in common. Preferably, the two sequences contain only up to 3, 5, 10, 15, or 20 amino acid insertions, deletions, and/or substitutions when compared to the other.

An “oligonucleotide”, as used herein, is a molecule comprising 2 to about 100 contiguous nucleotides linked via nucleotide bonds. An oligonucleotide may be at least about 10, 20, 30, 40, 50, or 60 nucleotides long. In addition, an oligonucleotide is preferably up to about 60, and more preferably up to about 50, 40, 30, or 20 nucleotides long.

A hybridization “probe” is an oligonucleotide that binds in a base-specific manner to a complementary strand of nucleic acid. Such probes include peptide nucleic acids, as described in Nielsen et al., 1991. Probes can be any length suitable for specific hybridization to the target nucleic acid sequence. The most appropriate length of the probe may vary depending upon the hybridization method in which it is being used; for example, particular lengths may be more appropriate for use in microfabricated arrays, while other lengths may be more suitable for use in classical hybridization methods. Such optimizations are known to the skilled artisan. Suitable probes and primers typically range from about 8 nucleotides to about 100 nucleotides in length. For example, probes and primers can be about 8-20, 10-30, 15-40, 50-80, and preferably 12-20 nucleotides in length. The nucleotide sequence can correspond to the coding sequence of the gene or to the complement of the coding sequence of the gene.

The Psoriasis Gene

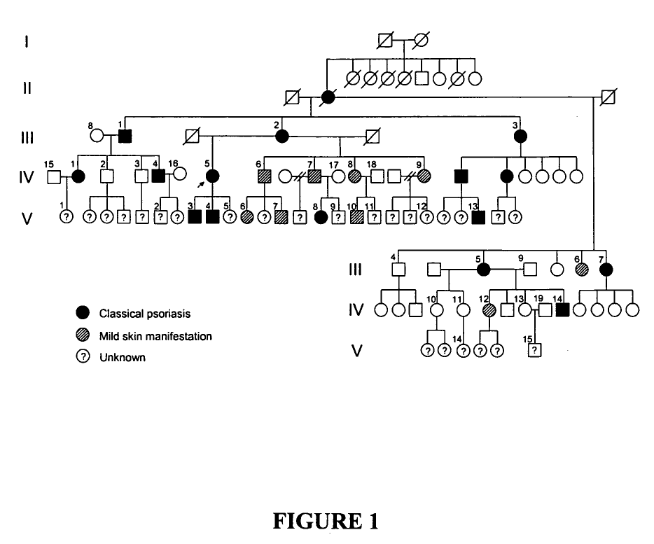

A five-generation family with psoriasis/PRP was employed to map the psoriasis/PRP gene. As described in Example 1 and FIG. 1, psoriasis/PRP occurred in this family in an autosomal dominant inheritance pattern. A series of linkage analyses was conducted to locate the gene to the terminal region of chromosome 17q (Example 2). Upon sequencing 8 candidate genes in this region, it was found that a point mutation in the CD7 gene correlated with psoriasis (Example 3). Therefore, CD7 is the gene that is responsible for psoriasis/PRP in this family. The point mutation was a C to T nucleotide change at position 658 of the CD7 cDNA, resulting in a single amino acid substitution, from Ala to Val, at amino acid residue 201 of CD7. This single nucleotide polymorphism (SNP) is thus associate with psoriasis/PRP.

CD7 is a member of the immunoglobulin superfamily. The sequence for human CD7 is shown below (SEQ ID NO:1; Accession Number gi|5453613):

| | |
|---|---|
| MAGPPRLLLL PLLLALARGL PGALAAQEVQ QSPHCTTVPV GASVNITCST SGGLRGIYLR.. | 60 |
| QLGPQPQDTI YYEDGVVPTT DRRFRGRIDF SGSQDNLTIT MHRLQLSDTG TYTCQAITEV | 120 |
| NVYGSGTLVL VTEEQSQGWH RCSDAPPRAS ALPAPPTGSA LPDPQTASAL PDPPAASALP. | 180 |
| AALAVISFLL GLGLGVACVL ARTQIKKLCS WRDKNSAACV VYEDMSHSRC NTLSSPNQYQ. | 240 |

CD7 is a Type I transmembrane glycoprotein of about 40 kDa. The signal peptide runs from residue 1 to approximately residue 25, an immunoglobulin domain variable region (IGv) is located between residues 37 and 132, and the transmembrane domain is near the C-terminus.

The present invention provides CD7 as a gene for psoriasis or its related disease pityriasis rubra pilaris (PRP). Also provided is a mutant CD7 comprising a mutation that is associated with psoriasis or pityriasis rubra pilaris (PRP). The polypeptide will be useful, for example, as immunogens for the preparation of antibodies that recognize the mutant CD7, or as a target protein in the screening of therapeutic agents for psoriasis. The polypeptide should comprise the relatively unique region of CD7, such as the C-terminal portion (approximately amino acid residues 133-240 of SEQ ID NO:1), and in particular residues 160-240 or 195-240 of SEQ ID NO:1. The polypeptide preferably comprises the entire mature CD7 molecule (residues 26-240) or the entire SEQ ID NO:1 except for the mutation.

The mutant CD7 should share a substantial amino acid sequence identity with SEQ ID NO:1. Its association with psoriasis or PRP can be determined as described herein, or by any method established in the art.

The polypeptide may be modified, such as glycosylated. The polypeptide may also be a fusion protein comprising sequences derived from a protein unrelated to CD7. For example, the fusion protein may comprise a detectable marker (e.g., luciferase, the green fluorescence protein, etc.) or a dimerization domain (e.g., a coiled-coil domain).

The mutant CD7 preferably comprises a mutation at amino acid 201. The mutation at amino acid residue 201 may be any insertion, deletion or substitution. Preferably, the mutation is a substitution, more preferably a substitution with valine.

The present invention also provides nucleic acids comprising a sequence that encodes the mutant CD7 described above. The nucleic acid may be, for example, cDNA or RNA. The nucleic acid may be a linear fragment, plasmid, cosmid, phage, virus or any other form known in the art. Preferably, the nucleic acid is an expression vector. Suitable expression vectors comprise a promoter that is active in the cells of interest. Expression vectors useful for practicing the present invention may also include selectable markers, cell-type or cell-cycle-specific enhancers or repressors, polylinkers, start codons, ribosome binding sites, internal ribosome entry sites, introns, stop codons, polyadenylation signals, or other features that facilitate cloning and vector stability, mRNA stability and localization in the cell, translation efficiency, or combinations thereof.

Cells harboring the nucleic acids described above are also provided. Preferably, the cells comprise an expression vector that contains a nucleic acid of interest. The cells may be eukaryotic or prokaryotic cells, including, without being limited to, bacteria, yeast, insect, avian and mammalian cells.

Of particular interest are oligonucleotides that are capable of hybridizing to the mutated CD7 gene but not the wild-type gene. The oligonucleotide will be useful, for example, as a hybridization probe or primer for the detection of the mutation in CD7, in particular the SNP wherein residue 201 is substituted with valine. The oligonucleotide preferably comprises a sequence that encodes Val-Leu-Val-Arg-Thr. More preferably, the oligonucleotide comprises GTGCTGGTGAGGACA (SEQ ID NO:3). For the purpose of hybridization, it is preferable that the sequence encoding the mutated amino acid residue 201 is near the center of the oligonucleotide. Preferably, there are at least 4 nucleotides on each side of the sequence encoding the mutated residue 201. For example, if the mutated residue 201 is valine, the codon for valine, or its complement, is preferably flanked by at least 4 additional nucleotides on each side. The codon is more preferably flanked by at least 5, 6, 7, 8 or 9 nucleotides on each side.

Hybridizations are usually performed under stringent conditions, for instance, at a salt concentration of no more than 1 M and a temperature of at least 25° C. For example, conditions of 5×SSPE (750 mM NaCl, mM NaPhosphate, mM EDT A, pH 7.4) and a temperature of 25-30° C., or equivalent conditions thereof, are suitable for single nucleotide-specific probe hybridizations. More preferably, a low stringent wash after hybridization is conducted, which includes, for example, 42° C., 5×SSC, and 0.1% SDS; or 50° C., 2×SSC, and 0.1% SDS. High-stringent wash conditions are most preferable and include, for example, 65° C., 0.1×SSC, and 0.1% SDS. Equivalent conditions can be determined by varying one or more of the parameters, as known in the art, while maintaining a similar degree of identity or similarity between the target nucleotide sequence and the primer or probe used.

The oligonucleotide can optionally be packaged in a kit. Depending on the purpose of the kit, the kit may comprise at least one additional component, such as one selected from the following group:
Hybridization equipments and/or reagents;
PCR equipments and/or reagents;
Primer extension reagents;
Equipments and/or reagents for collecting peripheral blood;
Other oligonucleotide primers or probes, such as those for amplifying the nucleic acid sequences around position 658 in the CD7 gene;
Instruction of use; and
Container for reagents.

The present invention further provides antibodies that recognize the mutant CD7 molecule, but not the wild-type. Methods for generating antibodies are known in the art (see, e.g., Harlow and Lane, 1989). Polypeptides or oligopeptides covering the area of interest (e.g., amino acid residues 133-240, 160-240, 195-240) can be used to raise antibodies against the mutant CD7 molecule. In particular, an oligopeptides covering a region of at least 7 amino acids, including the mutated residue 201, can be used as a hapten and conjugated to a carrier molecule for antibody production. Immunoselection or depletion methods can then be applied to ensure that the resulting antibodies are specific for the mutant and do not recognize the wild-type. For example, the resulting antibodies can be passed through an affinity column containing the wild-type CD7 molecule in order to remove antibodies that recognize the wild-type. Alternatively, antibodies, particularly monoclonal antibodies, can be screened using both the wild-type and mutant CD7 to identify those with the desired specificity.

In the present invention, we have mapped the psoriasis/PRP susceptibility locus to the extreme distal end of chromosome 17q. No significant linkage to other chromosomes, including 6p21, 10q, 16q and other loci previously reported for psoriasis, was evident in our analyses.

PSORS2 at 17q25 was one of the earliest identified psoriasis loci (Tomfohrde et al., 1994). In that study of eight Caucasian families, family 1 (PSI, 19 affected and 12 unaffected members) revealed strong linkage to D17S784 with a maximal two-point LOD score of 5.33 under a recombination fraction of 0.04. Family 4 (PS4, 9 affected and 12 unaffected) also displayed a significant linkage. Other families were smaller in size, and three families (PS6, 7 and 8) were not linked to D17S784. The investigators proposed that the psoriasis susceptibility gene lay within an 11 cM interval between D17S784 and D17S928 (which is an interval of 2.4 Mb, as calculated from the recently published human genomic sequence) (Tomfohrde et al., 1994).

Two subsequent studies confirmed the psoriasis susceptibility locus at 17q, although the linkage was not strong. Nair et al. found a LOD score of 2.09 in 726 individuals from nuclear or medium-size extended families from the United States and Germany (Nair et al., 1997). Enlund et al. demonstrated weak evidence of linkage to the locus on 17q, with a maximum NPL value of 1.83 (p=0.03) at D17S784, using a family set consisting of 104 families (153 sib pairs) from Sweden (Enlund et al., 1999a).

Recently, in the absence of significant linkage to microsatellite markers in the genotyping data from 242 nuclear families, non-parametric analysis performed with the GENEHUNTER program provided evidence for linkage to D17S1301 and a nearby immunoglobulin superfamily gene cluster (Speckman et al., 2003). Their follow-up study demonstrated two peaks of strong association with psoriasis separated by 6 Mb (Helms et al., 2003). The proximal peak is a putative RUNX1 binding site variant between SLC9A3R1 and NAT9, 8 Mb proximal to 17q terminus, which may be associated with susceptibility to psoriasis. The distal peak was not characterized in that study.

The studies described above were the sole source of published fine mapping data for PSORS2. The locus on 17q terminus that we identified herein is distal to and distinct from the previously reported locus. The discrepancy of the precise localization of the psoriasis susceptibility locus on chromosome 17q25 could be due to our use of a different study design. We used a single, large, extended family, while previous studies examined multiple, smaller families. Alternatively, the discrepancy could be simply due to genetic heterogeneity. As a third possibility, it is conceivable that the family studied herein was somehow unique in their presentation of psoriasis. With respect to the latter possibility, it may be germane that the clinical manifestations included not only both plaque psoriasis and erythrodermic psoriasis, but also very mild skin lesions and histopathologic conditions reminiscent of pityriasis rubra pilaris (PRP). Locating the psoriasis susceptibility gene in this family could thus shed light on the pathogenic processes of both psoriasis and PRP.

In this study, SNP#206 (0.38 Mb distal to D17S928) exhibited a strong association with psoriasis. SNP#206 is located at an intronic region of the tubulin-specific chaperone d (TBCD) gene (MIM *604649). TBCD is one of the tubulin-specific chaperone proteins responsible for microtubule assembly and regulation (Bhamidipati et al., 2000), and is widely expressed in tissues such as the skin. One of its partners, tubulin-specific chaperone cofactor C, may functionally overlap with the retinitis pigmentosa 2 protein (Bartolini et al., 2002). It is possible that a mutation or polymorphism in TBCD could alter the integrity of keratinocytes, and hence preclude the inflammatory change that is a hallmark of psoriasis. Therefore, TBCD, including its mutants and alleles, can be used as a target to screen drugs for psoriasis/PRP as well.

The present invention provides a method for assessing the risk for psoriasis or RPR in a subject, by examining the CD7 gene or gene product of the subject. If CD7 contains a mutation that is associated with psoriasis or pityriasis rubra pilaris (PRP), particularly a mutation at amino acid residue 201, the subject is at risk for psoriasis or RPR. The mutation is preferably an amino acid substitution at residue 201, more preferably an amino acid substitution with valine. The mutation can be examined by any method known in the art. In a preferred embodiment, genomic DNA is obtained from the subject, and the region around the mutation (e.g., nucleotides 657-659 for the mutation at residue 201) of the CD7 gene is amplified. Preferably, the amplification is performed using a primer that hybridizes to the mutated CD7 gene but not the wild-type. Therefore, if an amplification product can be observed after the amplification process, the subject has the mutated CD7 gene. Alternatively, the region around the mutation can be amplified using primers that do not include the mutated nucleotide(s), and the amplification product can be examined with a probe that hybridizes to the mutant CD7 gene but not the wild-type. In this latter approach, several probes, each of which is specific for a different kind of mutation of the mutated nucleotide(s), can be hybridized to the amplification product at the same time.

Another useful technique is the PCR-SSCP (single-strand conformation polymorphism) method (lizuka, et al. 1992; Mashiyama, et al. 1991; Iwahana, et al. 1995). This method is particularly preferable for screening many DNA samples, since it is relatively simple to operate, and only a small amount of test sample is required. The principle of the method is as follows. A single stranded DNA adopts a unique conformation that is dependent on its specific nucleotide sequence. After electrophoresis on a polyacrylamide gel without a denaturant, single-stranded DNAs having the same chain length shift to different positions in accordance with their respective conformation. The conformation of a single-stranded DNA changes with even a single base substitution, which results in a different mobility on polyacrylamide gel electrophoresis. Accordingly, the presence of a mutation in a DNA fragment can be detected based on the change in mobility, even if the mutation is only a single base substitution, deletion, or insertion.

By way of example, a region containing positions 657-659 in the CD7 gene can be first amplified by, for example, PCR. Preferably, a length of about 100 to 600 bp is amplified. The resulting DNA fragments can be labeled by using primers which are labeled, or by using substrate nucleotides which are labeled. Alternatively, the amplified fragment can be labeled afterwards, for example, by using the Klenow enzyme and labeled nucleotides. The labeled DNA fragments are then denatured and subjected to electrophoresis in a non-denaturing polyacrylamide gel. The condition for separating DNA fragments can be improved by adding appropriate amounts (about 5 to 10%) of glycerol to the polyacrylamide gel. Although the condition for electrophoresis varies depending on the characteristics of the DNA fragments, it is usually carried out at room temperature (20 to 25° C.). In the event separation is not satisfying at this temperature, the temperature may be varied between 4 to 30° C. After electrophoresis, the mobility of the DNA fragments is detected by means of the label or by staining the gel. When a band with a different mobility from the normal control is detected, the presence of a mutation can be confirmed by directly excising the band from the gel, amplifying it again by PCR, and sequencing the amplified fragment.

The denaturant gradient gel electrophoresis method (DGGE method) can also be employed. For example, a region containing positions 657-659 of the CD7 gene is amplified, electrophoresed on a polyacrylamide gel with a gradient of denaturant (e.g., urea), and the result is compared with that of a wild-type control. In the gradient gel, each sample begins to denature at a certain concentration of the denaturant, and mobility of the DNA decreases significantly at this point. Since the concentration at which each sample begins to denature depends on the sequence of the sample, the presence of a mutation can be detected by the difference in its position in the gel.

The allele specific oligonucleotide (ASO) hybridization method can be also used. An oligonucleotide is prepared from the test sample and subjected to hybridization with a specific probe. If the oligonucleotide contains a nucleotide that is different from those in the probe, the efficiency of hybridization is reduced. The decrease in hybridization efficiency can be detected by Southern blotting, specific fluorescent reagents that are quenched by intercalation into any mismatch in a hybrid, or any other methods available in the art.

The detection may also be performed by the ribonuclease A mismatch truncation method. Specifically, if the mutation is at residue 201, a region containing positions 657-659 of the CD7 gene is amplified, and the amplified products are hybridized with labeled RNAs having the wild-type sequence. If the sample contains a mutation, there would be a mismatch in the hybrid, which can be cleaved by ribonuclease A. The reaction mixture is then resolved by electrophoresis, and a mutant sample can be identified as containing a shorter fragment that results from the cleavage.

In addition to CD7, SNP#206 can also be used as a marker in the prediction, diagnosis, and disease monitoring of psoriasis or PRP. SNP#206 can be detected according to any method, such as the ones discussed above.

Our finding of CD7 as a psoriasis gene is consistent with the notion that psoriasis is primarily not a disorder of keratocyte proliferation, but an immune-mediated disorder dependent on T-cell activation (International J Dermatol 41:827, 2002). Recent therapeutic development for psoriasis has focused on monoclonal antibody therapy directed against key components of the inflammatory process. An anti-CD2 monoclonal antibody (Amevive, Biogen Inc) has been approved this year by the FDA, and an anti-CD11a antibody (Raptiva, Genetech/Xoma) is in phase III clinical trial for moderate-to-severe psoriasis. However, not all patients respond to these antibodies.

The present invention provides CD7, as well as its associated pathways/proteins, as a new target for psoriasis and PRP therapeutic research and development. CD7 is expressed in prothymocytes and persists through the development of T cells. NK cells also express CD7. Similar to CD28, CD7 has been demonstrated to act as a costimulatory molecule (Stillwell et al., 2001). Crosslinking with anti-hCD7 monoclonal antibody has been reported to be mitogenic (Carrera et al., 1988), augment IL-2 production (Ledbetter et al., 1987), and increase calcium fluxes (Jung et al., 1992) as well as integrin-mediated adhesion (Chan et al., 1997). CD7 can be associated with CD3, CD45 (Lazarovits et al., 1994), P13-kinase (phosphatidylinositol 3-kinase) (Chan et al., 1997) and type II P14-kinase (Subrahmanyam et al., 2003), highlighting the importance of CD7-mediated signaling in T cells.

It has been demonstrated that CD7 is able to bind human galectin-1, galectin-3, and the K12 (SECTM1) gene product. Galectin-1 and galectin-3 are expressed in a variety of cells and tissues, and may be important mediators of immune regulation. After binding to the cell surface through CD7 and CD45, extracellular galectin-1 and galectin-3 can induce apoptosis of thymocytes and mature T cells (Fukumori et al., 2003; Roberts et al., 2003). K12, located just upstream of the CD7 locus, has been shown to increase surface expression of NK cell activation markers (Lyman et al., 2000).

It is contemplated that the CD7 mutants identified herein possess altered activities and over-stimulate the immune system, particularly the inflammatory cells. Thus, for example, cells expressing a CD7 mutant of the present invention can be prepared. Candidate drugs can be added to the cell, and their impacts on a CD7 downstream effect, e.g., tyrosine phosphorylation, are measured and compared to the results obtained from control cells expressing the wild-type CD7. The drugs capable of restoring the activity of the CD7 mutant to that of the wild-type are potential psoriasis and PRP drugs, and can be further developed and tested. Other screening methods using CD7 or its associated pathway/proteins can be designed based on ordinary skill in the art, the disclosure herein, as well as knowledge available in the literature.

The present invention further provides a method for pharmacogenomic profiling. Thus, a panel of genetic factors is determined for a given individual, and each genetic factor is associated with the predisposition for a disease or medical condition, including psoriasis and PRP. In accordance with the present invention, CD7 and/or SNP#206 are included as markers for psoriasis and PRP. The panel may optionally include other genetic factors for psoriasis and PRP, such as the RUNX1 variant reported by (Helms et al., 2003). In addition to markers for psoriasis and PRP, the panel may include any other known genetic factors, such as thiopurine methyltransferase and the genes for the long-QT syndrome.

In order to further illustrate the present invention and advantages thereof, the following specific examples are given but are not meant to limit the scope of the claims in any way.

### EXAMPLES

In the examples below, the following abbreviations have the following meanings. Abbreviations not defined have their generally accepted meanings.
° C.=degree Celsius
hr=hour
min=minute
sec or s=second
μM=micromolar
mM=millimolar
M=molar
ml=milliliter
μl=microliter
mg=milligram
μg=microgram
mol=mole
pmol=picomole
SNP=single nucleotide polymorphism
PBS=phosphate buffered saline
NTP=nucleoside triphosphate
PRP=pityriasis rubra pilaris
PCR=polymerase chain reaction
NPL=nonparametric linkage
SSCP=single strand conformation polymorphism
DGGE=gradient gel electrophoresis
ASO=allele specific oligonucleotide
   Materials and Methods
   Family Ascertainment

A five-generation psoriasis kindred with an apparent autosomal dominant inheritance pattern (FIG. 1) was identified at the National Taiwan University Hospital. All individuals were examined by at least two dermatologists. This family comprised a total of 93 members, including 16 with classical skin manifestations of psoriasis, 10 with mild skin lesions, 43 unaffected, and 24 with an unknown disease status. In this family, the severity of the disease varied, ranging from mild skin lesions resembling PRP to severe classical psoriasis. Because of the age-dependent variation in the expression of psoriasis/PRP, family members aged less than 30 who appeared normal were designated with the “unknown disease” status.

DNA samples were available from 43 individuals (those numbered in FIG. 1). The study was approved by the institutional review board at the National Taiwan University Hospital. Informed consent was obtained from each participating person.

Genotyping

Genomic DNA was extracted using the Puregene DNA Isolation Kit (Gentra Systems, Inc., Minneapolis, Minn., USA) following the manufacturer's protocols. The DNA samples were stored at −70° C. until genotyping was undertaken.

Genotyping was performed for 382 highly polymorphic microsatellite markers from the ABI PRISM® Linkage Mapping Set v 2.5 HD5 (Applied Biosystems, Foster City, Calif., USA). The average heterozygosity of markers was 0.72% with an estimated 10 cM spacing. Polymerase chain reactions (PCR) for genotyping were performed using a Hamilton robot (Microlab 4000 series, Westburg, Lensden, Netherlands) and the GeneAmp 9700 thermocyclers (Applied Biosystems). All PCR reactions were performed in a 5° ml volume containing 10 ng of genomic DNA and 0.33 mM of each primer. Each marker was investigated in an individual PCR, and up to 20 reactions containing appropriate sizes and fluorescent labels were pooled for gel electrophoresis analysis. The PCR products were separated using the ABI 3730 DNA sequencer, allowing multiple fluorescently labeled markers to be run in a single lane (Applied Biosystems). The LIZ500 size standard was run as an internal size standard (Applied Biosystems). Allele sizing was calculated using GENOMAPPER software program (Applied Biosystems). Allele calling and binning were performed using SAS program (SAS institute Inc.). All genotyping was performed with the inclusion of four CEPH (The Centre d'Etude du Polymorphisme Humain (http://landru.cephb.fr/)) control individuals (1331-01, 1331-02, 1347 02, H20) for quality control purposes.

In order to improve the resolution of mapping on 17q25, six additional markers between D17S785 and D17S784 were selected for genotying from the Genethon, Cooperative Human Linkage Center (CHLC; http://gai.nci.nih.gov/CHLC/), and the Marshfield Clinic Research Foundation (Marshfield, Wis., USA; http://www.marshfieldclinic.org). The order and positions of the markers, as obtained from the Marshieldfield Genetic Database, were D17S801(75.1 Mb)—D17S722(75.59 Mb)—D17S939(76.05 Mb)—D17S802(76.83 Mb)—D17S1806(78.04 Mb)—D17S1822(78.48 Mb).

Single Nucleotide Polymorphism Selection and Typing

High-density single nucleotide polymorphisms (SNPs) with minor allele frequency between 0.1 and 0.5 were selected from the SNP Consortium (http://snp.cshl.org/) and the NCBI website (http://www.ncbi.nlm.nih.gov/). Primers and probes that flanked the SNPs were designed in multiplex format using the SpectroDESIGNER software (Sequenom, San Diego, Calif., USA). Multiplex PCR was performed in a 5° μl reaction volume containing 0.15 units of Taq polymerase (HotStarTaq™, Qiagen, Valencia, Calif., USA), 5.0 ng genomic DNA, 1.0 pmol of each PCR primer, and 2.5 nmol of dNTP. Thermocycling consisted of one cycle at 94° C. for 15 min, 45 cycles of 94° C for 20 s, 56° C. for 30 s, 72° C. for 30 s, and one final cycle of extension at 72° C. for 3 min. Unincorporated dNTPs were dephosphorylated using 0.3 U of Shrimp Alkaline Phosphatase (Hoffman-LaRoche, Basel, Switzerland) followed by primer extension using 9 pmol of each primer extension probe, 4.5 nmole of the appropriate dNTP/ddNTP combination, and 1.28 units of Thermosequenase (Amersham Pharmacia, Piscataway, N.J., USA). The reactions were cycled at 94° C. for 2 min, followed by 55 cycles of 94° C. for 5 s, 52° C. for 5 s, and 72° C. for 5 s. Following the addition of a cation exchange resin (SpectroCLEAN, Sequenom, San Diego, Calif.) to remove residual salt from the reactions, 15 nl of the purified primer extension reaction was spotted onto a 384-element silicon chip preloaded with 3-hydroxypicoloinic acid matrix (SpectroCHIP, Sequenom) using the SpectroPOINT (Sequenom). The SpectroCHIPs were analyzed using a Bruker Biflex III MALDI-TOF SpectroREADER mass spectrometer (Sequenom) and spectra processed using SpectroTYPER (Sequenom).

Statistical Analyses

Mendelian inheritance for the 382 microsatellite markers of this pedigree was verified by use of the SAS/Genetics program. The allele frequencies were estimated by using 90 individuals randomly chosen from our previously collected control population. Two point analyses for these markers were first calculated using the MLINK program of the LINKAGE package (http://linkage.rockefeller.edu/). For the most significant results obtained in the region covering D17S785, D17S784, and D17S928, multipoint analysis using the LINKMAP program (http://linkage.rockefeller.edu/) was carried out by incorporating the six additional microsatellite markers described above. An autosomal dominant model with 80% penetrance and 0.1% phenocopy rate was assumed for the calculations. The disease gene frequency was 0.00062, assuming a prevalence of 0.0012.

Two point analyses were also performed for the SNP markers in this region. Multipoint analysis was performed using the SIMWALK2 program (http:/Hlinkage.rockefeller.edu/). A modified version of the PDT program (Martin et al., 2001), which utilizes a permutation test to calculate empirical p-value, was used to test the allelic association between the SNP markers and psoriasis. In calculating the empirical p-value, 10,000 permutations were carried out for each SNP.

### Example 1

### A Five-Generation Family with Psoriasis

A five-generation family was studied to map the genes associated with psoriasis/PRP. FIG. 1 shows the pedigree of this family and the disease state of each family member. The age of onset of psoriasis/PRP and the severity of the disease varied in the family members. For example, the proband (individual IV-5, indicated by an arrow in FIG. 1), a 41-year-old woman, showed plaque-like scaling skin lesions with crackling and fissuring over most of her skin surface, including the palms and scalp. Her condition had developed aggressively and periodically since the age of 10. Onycholysis and arthritis were present during flare-up of the disease. However, her son (V-4) exhibited erythrodermic psoriasis with generalized, inflamed erythematous and fever.

Variation in phenotypes also occurred between other consecutive generations, for example in the IV-7 father/V-8 daughter pair. Well-demarcated, pink-to-red colored skin lesions had been noted in V-8 since the age of three. The palm-sized lesions of V-8 were thinner and less scaly compared to the psoriasis plaques seen in the adults, but they extended from her extremities to the trunk within six months. IV-7 and some other family members exhibited mild, ichthyosis-like skin lesions over their legs that were usually stable for years. Lesions in III-1 -were only visible over the extensor side of the knuckles, elbows and knees. As the patients aged, their skin became diffusely atrophic as in III-2, which appeared unrelated to the use of corticosteroid.

No triggering factors were identified except that the proband complained about an aggravation of her disease during pregnancy. Arthritis was not a major symptom in this family, but some patients had deformed finger joints. There is no gender difference in the number affected, disease severity, or transmission rate.

Skin biopsy specimens from V-4 revealed marked hyperkeratosis and parakeratosis with follicular plugging, loss of granular layer, epidermal acathosis and elongation of rete ridges, thin suprapapillary plates, vascular dilatation, inflammatory cell infiltration in the dermis and epidermis, and a suspicious Munro's microabscess. Histological changes in V-8 were similar but much less severe. Of particular interest was the presence of alternating orthokeratosis and parakeratosis, a histopathologic condition reminiscent of pityriasis rubra pilaris (PRP).

### Example 2

### Linkage Analysis

We performed a genome-wide scan with polymorphic microsatellite markers in the five-generation psoriasis kindred described in Example 1. A total of 382 markers were employed, which distributed across the 22 autosomes with an average intermarker distance of 10 cM. The genotyping analysis included 43 DNA samples from 13 individuals with classical skin manifestations of psoriasis, 10 with mild skin lesions, 7 with unknown disease status, and 13 family members who were unaffected.

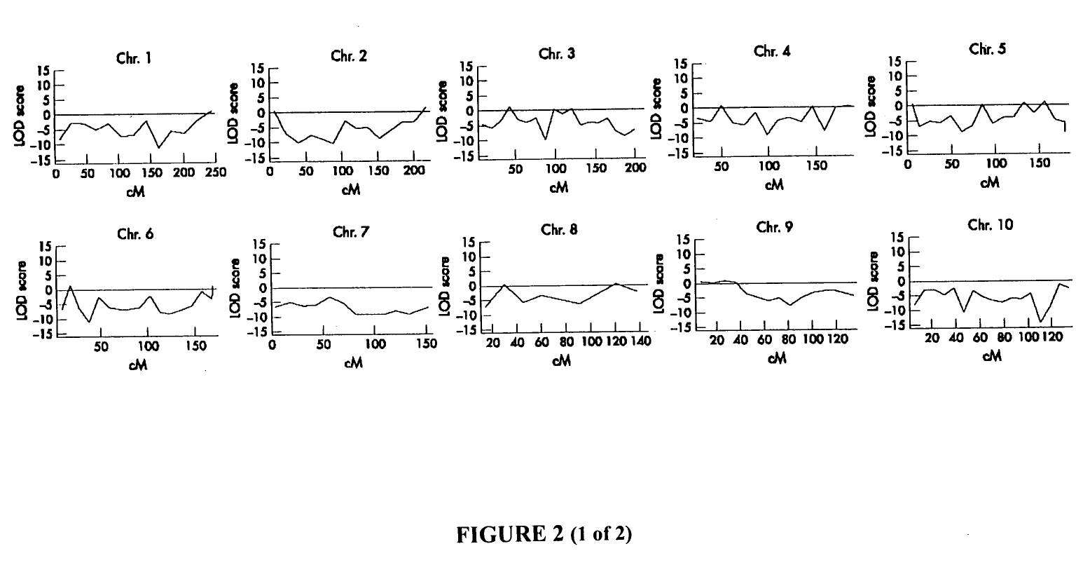

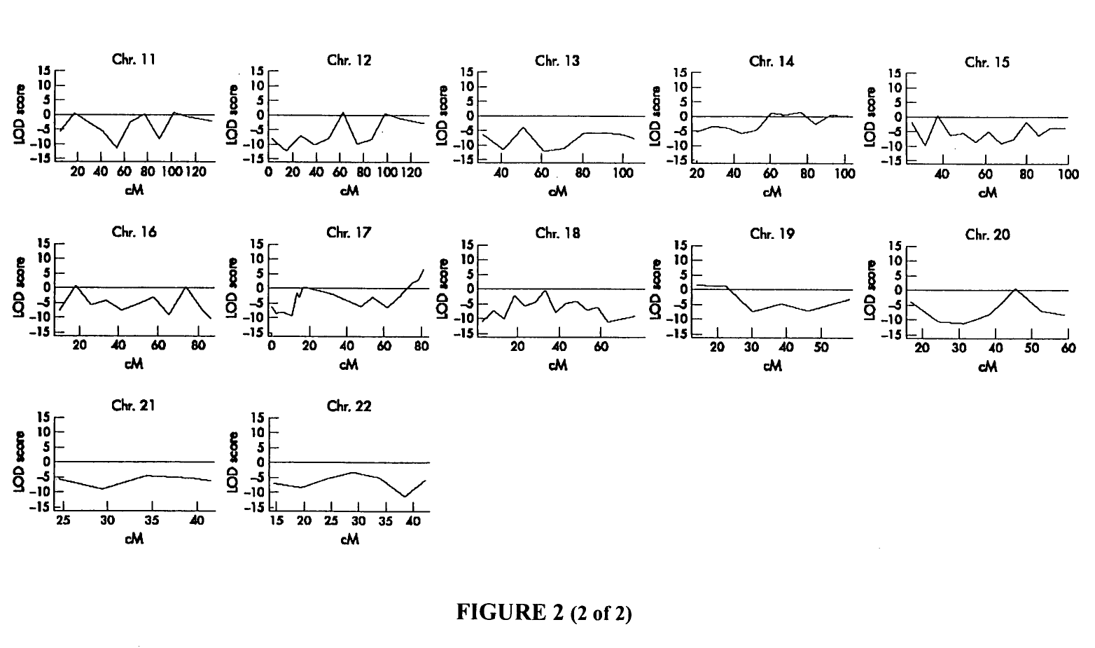

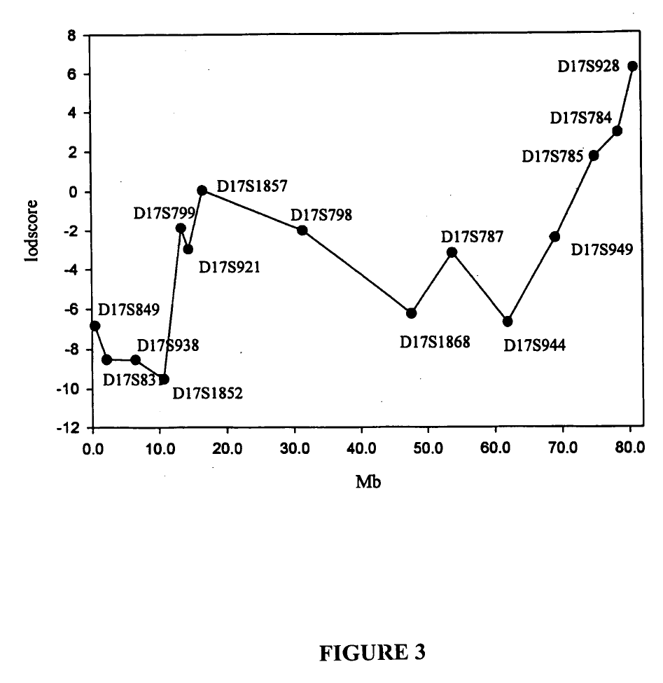

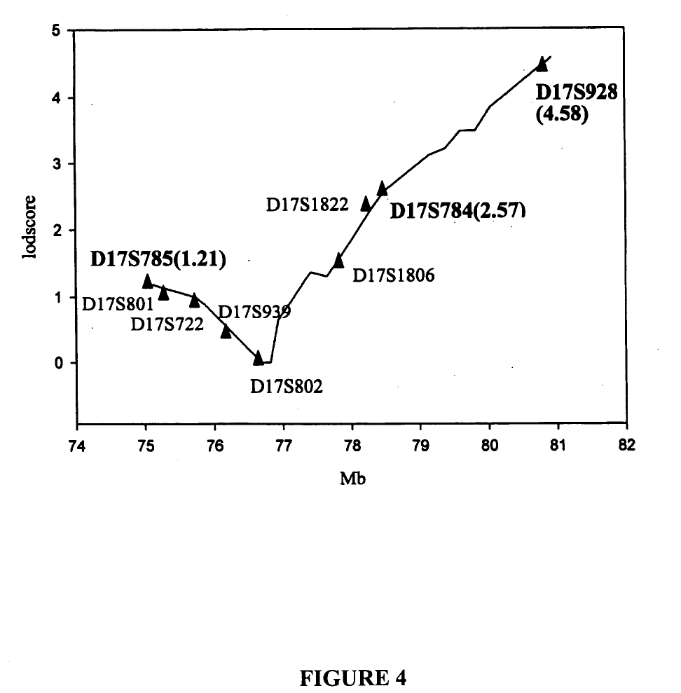

One region covering D17S785, D17S784 and D17S928 on chromosome 17q showed highly significant linkage with the disease, with a maximum two-point LOD score of 7.164 at D17S928 (q=0.01) (FIGS. 2 and 3). The LOD scores for markers over other regions of chromosome 17 and other chromosomes were not significant (FIG. 2). To further validate our results and narrow down the candidate region, six additional microsatellite markers between D17S785 and D17S784 were added (D17S801, D17S722, D17S939, D17S802, D17S1806 and D17S1822). Multipoint analysis using the LINKMAP program demonstrated a LOD score of 1.21 for D17S785, 2.57 for D17S784, and 4.58 for D17S928 (FIG. 4). These data are consistent with a gene location close or distal to D17S928, which is the most distal polymorphic microsatellite marker on 17q known to date. The distance between D17S928 and the end of 17q is around 800 kb.

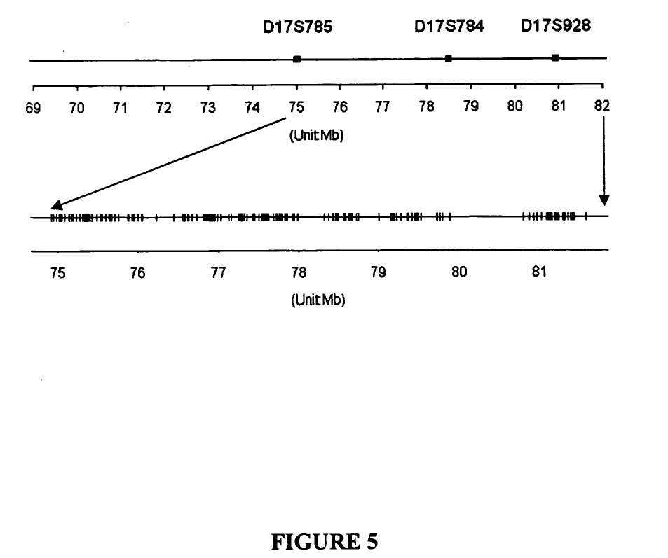

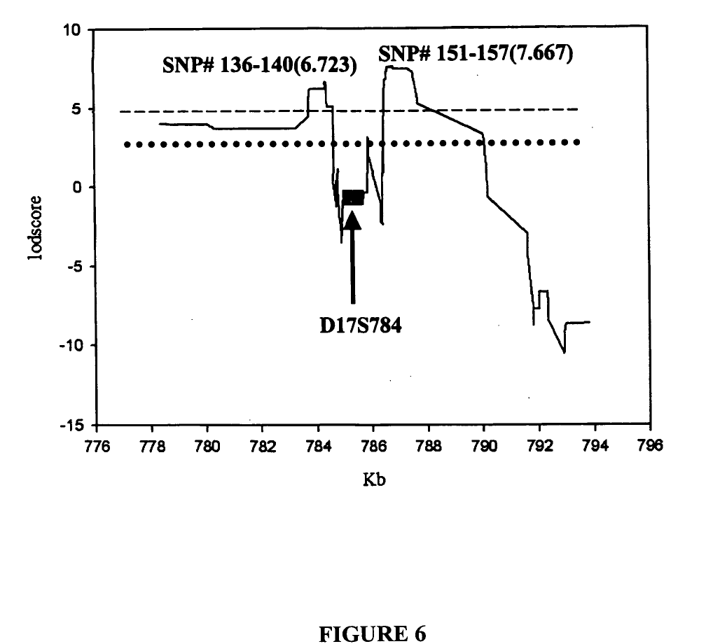

To fine-tune the mapping, we further genotyped a total of 202 SNP markers located between D17S785 and the 17q terminus (FIG. 5). A list of these markers are attached as Appendix A. The results from two-point linkage analysis indicated that the linkage peaked at SNP#152 with an LOD of 4.45, and at SNP#204 with an LOD of 4.47. Since the significance was toward areas around D17S784 and 17q terminus, two multipoint analyses using the SIMWALK2 program were performed using 38 SNP markers in the former, and 45 SNP in the latter region, respectively. Two plateaus of significant LOD score (>3.3) were yielded from the multipoint SNP analysis around the region of D17S784, with the highest LOD of 7.67 at SNP#154 and SNP#155. Around 17q terminus, the highest LOD of 3.304 appeared at SNP#211 (FIG. 6).

We further calculated the allelic associations of SNP markers in the two regions (29 markers, #130 to #158, around D17S784; 9 marker, #203 to #211, around 17q terminus) using a modified version of the PDT program. The variance for the PDT in the original program was obtained from more than one family. Since there is only a single family involved in our analysis, a permutation test was used to derive the empirical p-value. The results showed no significant association in the region from #130 to #158. However, in the region from #203 to #211, a significant association was identified between SNP#206 and psoriasis (p=0.0008), as shown in Table 1. This marker (rs3744165) is located at an intronic region of the tubulin-specific chaperone d (TBCD) gene (MIM *604649), approximately 400 kb from the 17q terminus.

TABLE 1
Allelic association between six SNP markers and psoriasis

| SNP Number | P value * |
|---|---|
| 203 | 0.156 |
| 204 | 0.215 |
| 205 | 0.135 |
| 206 | 0.0008 |
| 207 | 0.11 |
| 208 | 0.136 |
| 209 | 0.181 |
| 210 | 0.115 |
| 211 | 0.198 |

* The empirical P value was calculated from 10,000 permutations in the PDT program using PDT-average statistics.

### Example 3

### The Psoriasis Gene

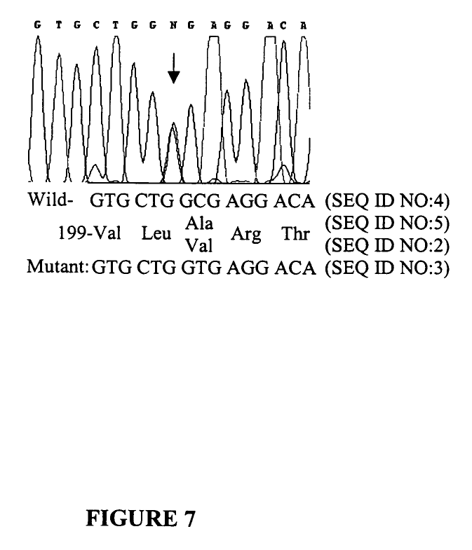

To identify the gene that is responsible for psoriasis/PRP in this family, we sequenced 8 candidate genes within the region identified in Example 2. The results show that CD7 is assoicated with the A heterozygous C to T nucleotide change at position 658 of the CD7 cDNA was found in all the affected family members, but not in the unaffected. Furthermore, this mutation was not found in any of 200 control subjects in the population. This point mutation changes amino acid 201 from alanine to valine (FIG. 7), which is located in the predicted transmembrane domain of the CD7 molecule. These results indicate that CD7 is the gene for psoriasis/PRP in this family.

While this invention had been particularly shown and described with references to preferred embodiments therefor, it will be understood by those skilled in the art that various changes in form and detailed may be made therein without departing from the spirit and scope of the invention as defined by the appended claims.

1. An isolated polypeptide comprising amino acid residues 133-240 of SEQ ID NO:1, except that amino acid residue 201 of SEQ ID NO:1 is mutated.

2. The polypeptide of claim 1 wherein amino acid residue 201 is substituted with valine.

3. The polypeptide of claim 1 consisting of SEQ ID NO:1, with amino acid residue 201 being mutated.

4. The polypeptide of claim 3 wherein amino acid residue 201 is substituted with valine.

5. The polypeptide of claim 1 that is glycosylated.

6. An isolated nucleic acid comprising the nucleotide sequence encoding the polypeptide of claim 1.

7. An expression vector comprising the nucleic acid of claim 6.

8. A cell comprising the expression vector of claim 7.

9. An oligonucleotide that hybridizes to a mutated human CD7 gene encoding a CD7 wherein amino acid residue 201 is mutated, but not the normal human CD7 gene.

10. The oligonucleotide of claim 9 wherein the mutated human CD 7 gene encodes a CD7 wherein amino acid residue 201 is valine.

11. The oligonucleotide of claim 9 comprising a sequence that encodes Val-Leu-Val-Arg-Thr, or the complement thereof.

12. The oligonucleotide of claim 11 wherein the sequence is GTGCTGGTGAGGACA (SEQ ID NO:3) or the complement thereof.

13. The oligonucleotide of claim 9 consisting of about 20 nucleotides or less.

14. The oligonucleotide of claim 9 consisting of about 30 nucleotides or less.

15. The oligonucleotide of claim 9 consisting of about 50 nucleotides or less.

16. A kit comprising the oligonucleotide of claim 9.

17. An array of oligonucleotides comprising the oligonucleotide of claim 9.

18. An isolated antibody that recognizes the polypeptide of claim 3 but not SEQ ID NO:1.

19. The antibody of claim 18 wherein the polypeptide of claim 3 contains valine at amino acid residue 201.

20. A method for assessing the risk for developing psoriasis or pityriasis rubra pilaris (PRP) in a subject, comprising examining the CD7 gene or gene product of the subject, wherein a mutation in the CD7 gene is indicative of a risk for developing psoriasis or PRP.

21. The method of claim 20 wherein the mutation is at amino acid 201 of CD7.

22. The method of claim 21 wherein the mutation is an amino acid substitution.

23. The method of claim 21 wherein amino acid residue 201 of CD7 is substituted with valine.

24. The method of claim 20 wherein the mutation is detected by using genomic DNA obtained from the subject.

25. The method of claim 24 wherein the genomic DNA is obtained from the blood of the subject.

26. The method of claim 20 wherein the mutation is detected by polymerase chain reaction.

27. A method for developing a therapy for psoriasis or pityriasis rubra pilaris (PRP), comprising screening candidate medicines using an assay in which CD7 or a CD7 mutant is the target.

28. The method of claim 27 wherein the CD7 mutant comprises a mutation at amino acid residue 201.

29. The method of claim 28 wherein the mutation is an amino acid substitution.

30. The method of claim 28 wherein the mutation is a substitution with valine.

31. The method of claim 27 performed by using a cell that expresses the CD7 mutant.
